(19) United States
(12) Patent Application Publication
(10) Pub. No.: US 2008/0038326 A1
(43) Pub. Date: Feb. 14, 2008

(54) Multilayered Dosing Systems

(76) Inventors: Bruce Michael Drew, Great Dunmow (GB); Shilpan Patel, Mumbai (IN); Guy Friedman, Tel-Aviv (IL)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 11/793,956

(22) PCT Filed: Dec. 23, 2005
(86) PCT No.: PCT/GB05/05090

§ 371 Date: Jun. 22, 2007

(30) Foreign Application Priority Data
Dec. 24, 2004 (GB) 0428226.5

(51) Int. Cl. A61K 9/70; A61K 47/36

(52) U.S. Cl. 424/444; 424/443; 514/778

(57) ABSTRACT

The invention relates to dosing systems which involve water-soluble or water dispersible products containing ingredients embedded within and/or coated upon a water-soluble film which are released at a prescribed rate into an aqueous environment through one or more perforated less readily soluble water-soluble films. The dosing system can be used for delivering pharmaceutical active ingredients externally or internally to a human or animal body, and can be embodied in the form of a patch that is applied to a site to be treated via a water soluble adhering layer (1) that adheres to the site with ingredients(s) provided by a carrier layer (3) sandwiched between a perforated layer (2) for delivery of the ingredients to the site and a non-perforated protecting layer (4).

The invention relates to dosing systems, and concerns in particular such systems which involve water-soluble or water dispersible products (as both terms are defined herein) containing ingredients embedded within and/or coated upon a water-soluble or water dispersible film which ingredients are released at a prescribed rate into an aqueous environment through one or more perforated less readily soluble water-soluble or water dispersible films. The invention relates especially, but not exclusively, to the use of such products in delivering pharmaceutical active ingredients externally or internally to a human or animal body.

As used herein, the term “water soluble” refers to materials that are capable of being dissolved in water of whatever temperature to form a homogeneous solution and the term “water dispersible” refers to materials that are capable of being dispersed in water of whatever temperature to form a permanent or temporary suspension. For convenience, where the term “water soluble” is used hereinafter in the description and claims, it will be understood this includes “water dispersible” and vice versa.

The ability to implant drug delivery systems within the human or animal body during a surgical intervention has been reliant on various treatments, all of which have had to be removed in a second surgical intervention once the drug(s) has been delivered and done its work. Such medications, whilst providing varying degrees of effectiveness, all share common limitations such as the inability to deliver the drug accurately and reliably to the site of the intervention for the optimum level of time and the need to remove the drug delivery system in a second intervention. Delivering a drug accurately and reliably to the site of the intervention for the optimum level of time has advantages compared to alternative methods of drug delivery such as intravenous injection, oral tablets, capsules or suppositories.

There are similar difficulties of delivering a drug accurately and reliably to the affected area for the optimum level of time in the treatment of open wounds on the surface of the body resulting, for example, from burns, scalds or injuries incurred during warfare. Here, the major difficulties are again the need to deliver the drug accurately and reliably to the affected area, in this case an open wound, and again, the removal of the drug delivery system after treatment without damage to the healing tissue.

The present invention is designed to address the limitations highlighted above.

According to a first aspect of the invention there is provided a water-soluble or water-dispersible product containing one or more ingredients in which the ingredient is embedded within or coated upon one or more water soluble films which will be described herein as carrier water-soluble films, which are protected by less readily soluble water-soluble films on either side, at least one of which is perforated to allow the migration of ingredient through the perforations as soon as the product is placed in contact with an aqueous medium at an appropriate site, thereby allowing the ingredient to be dispensed and the product to be at least partially dissolved or dispersed over a period of time such that there is no need for its subsequent physical removal from the site.

In considering the above invention, the term “aqueous medium” should be taken to mean any water-based liquid or gel environment including the different aqueous fluids of varying pH which are to be found within the body, within the gastro-intestinal tract, on the tongue, within the rectum or vagina, within the eye cavity, or within an open wound upon the surface of the body, wherein this term refers to the human or animal body.

The product can, in theory, be of any thickness. In practice, however, a thickness in the range 15 μm to 500 μm is generally preferred as products of thickness less than 15 μm may not be strong enough and those of thickness greater than 500 μm may be too stiff to be applied easily to the site to be treated.

Using this invention, the rate at which the ingredient(s) is released from the carrier water-soluble film can be accurately controlled in order that the ingredient(s) can be dispensed accurately and reliably by laminating—by any method—protecting water-soluble films to either side of the carrier water-soluble film wherein the protecting water-soluble films are less soluble at a given temperature than the carrier water-soluble film.

Any means of providing protecting water-soluble films which are less soluble than the carrier water-soluble film at a given temperature may be used.

Preferably, one of the protecting water-soluble films is perforated in order to allow the ingredient(s) to seep through the perforations over a prescribed period of time. It will be evident that the rate of release of the ingredient(s) is controlled by the solubility of the carrier water-soluble film and the size and number of perforations in the perforated protecting water-soluble film. As the aqueous medium seeps through the perforations, it progressively solubilises the carrier water-soluble film, thereby releasing the ingredient(s) to seep back through the perforations to be delivered to the site where the ingredient(s) are required.

Preferably, a relatively quickly dissolving water-soluble film is laminated over the perforated water soluble film that protects the carrier film from manufacture through to use and, in use, assists in attaching the product to the site to be treated. This film is described hereinafter as the “adhering” water-soluble film and is preferably made easily distinguishable from the unperforated protecting water-soluble film, in order to avoid the product being applied the wrong way round. A simple way to avoid such confusion, although this is by no means limiting, is for the “adhering” water-soluble film to be an embossed film to distinguish it from the unperforated protecting water-soluble film which is not an embossed film.

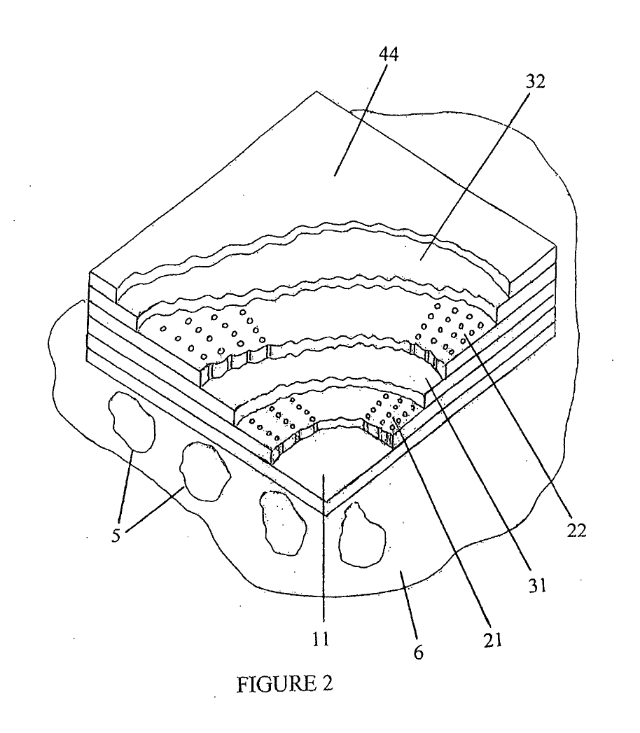

Thus, one embodiment of the invention concerns a drug delivery system in the form of a water soluble film, or laminate of two or more such films, or extrusion of one upon another such film, carrying on one or more of its surfaces or within the mass of one or more films comprising the laminate, one or more pharmaceutical active ingredients required to be delivered to an appropriate site such as a surgical intervention, the tongue, vagina, rectum, eye cavity or an open wound and which will self adhere to at least part of the site to be treated and which will be partially or totally dispersed into an aqueous medium present at the site such that there is no need for its subsequent physical removal from that site.

Where more than one active ingredient is required to be delivered, controlled release of different ingredients in stages can be obtained by increasing the number of carrier water-soluble films within the laminated product. The plurality of carrier water-soluble films containing different ingredients can either be laminated together or they can be separated by further perforated water-soluble films as can be seen in FIG. 2. In either case, they are presented to the site in such a way that the delivery sequence of ingredients is in the prescribed order. In this way, each ingredient becomes available at the appropriate time for optimum treatment.

The incorporation of one or more required ingredients into the mass of the carrier water-soluble film as opposed to printing or coating the ingredients on one or both surfaces of the carrier water-soluble film allows slow release presentations to be formulated by selecting lower solubility polymers from which to manufacture the carrier water-soluble film. By this means, the required active ingredients are made available over a prescribed period of time.

The thickness of the product can also be used to vary the rate of dispensing active ingredients with time in either of two ways:
1. Where the active ingredients are held on the surface of the carrier water-soluble film, the active ingredients may be released at such a rate that the product may disperse in the aqueous medium before the treatment is complete. This can be overcome by making the perforated protecting water-soluble film thicker and/or less soluble than the carrier water-soluble film at any given temperature.
2. Where the active ingredients are incorporated within the mass of the carrier water-soluble film, they are released at a steady rate as the carrier water-soluble film dissolves. The duration of the treatment can therefore be varied by adjusting the rate of solubilisation and the thickness of the carrier water-soluble film.

The presence of an adhering water-soluble film as part of the product causes an immediate adherence to the surface being treated, due to the presence of an aqueous medium at the site to be treated, whether this moisture is present due to bodily or other fluids or has deliberately been applied to the surface in preparation for the application of the product. The level of adherence can be varied by adjusting the composition and type of adhering water-soluble film; different adhering water-soluble film compositions can be selected for different site conditions. It will be understood that the adhering water-soluble film will automatically self adhere to moist surfaces due to the solubilisation of its surface producing an adhesive effect. In situations where the quantity of aqueous medium present on the surface is insufficient to solubilise enough of the surface area of the product to obtain self-adherence of the product, the self adherence may be supplemented or in the limit fully obtained by means of a water-soluble adhesive that has been either applied to the product during its manufacture or applied to the surface immediately prior to the application of the product. This invention can use any composition of adhering water-soluble film provided it forms a suitably adhesive surface with the amount of aqueous medium present at the site of application.

It is preferable, in the case where the moist surface to be treated forms part of the human or animal body, that the product is made from water-soluble films that have been approved for ingestion or absorption into body tissues in the application area to which use of the product is intended. Films that have been approved for absorption into body tissue are described herein as “bioabsorbable” where this term is defined as meaning those materials that are capable of being degraded in vivo by the natural action of the body so that they may be gradually absorbed into body tissue.

At the present time, hydroxy propyl methyl cellulose (HPMC), pullulan, or starch based films such as STARPOL® are preferred materials for the water soluble films of the invented product in bioabsorbable applications. However, these materials are by no means limiting. Whether for medical use or for other applications, other water-soluble materials can be used, as they become approved for each application, including those films based upon polyvinyl alcohol (PVOH), polyethylene oxide, modified celluloses such as carboxy-methyl cellulose (CMC), dextrin, guar gum, gelatine and their derivatives. In the case where one or more films is based upon PVOH, their solubility at a given temperature may be adjusted by selecting a grade with a specific molecular weight and a specific % saponification. By way of example only, to obtain solubilisation of a water-soluble film made substantially from PVOH in an aqueous medium at a temperature at or above 60° C., the PVOH should preferably be of a fully saponified type i.e. the degree of saponification ranging from 90-99.5%, and to obtain solubilisation in an aqueous medium at a temperature of 25° C. and below, the PVOH should be of a partially saponified type i.e. the degree of saponification ranging from 71-90%. Hence, the lower the degree of saponification, the lower the temperature of water required to dissolve a water soluble film made substantially from PVOH.

When the adhering water-soluble film is based upon PVOH, it is possible to increase its level of tack or stickiness by using film made from a lower molecular weight of PVOH than would otherwise be used. Additives like guar gum, dextrin, starch and gelatine compositions can be added to increase the relative tack value of the adhering water-soluble film. This may be important where the amount of aqueous medium at the site is low. Similarly, it is possible to increase the flexibility of the product in order to provide increased contact with the moist surface by using an adhering water-soluble film with a higher water content than the range of 4.0-8.0% found within most commercially available water-soluble films. An adhering water-soluble film having a water/moisture content of up to 14% may be used. Using different kinds of plasticisers can also affect the flexibility of the product as well as the relative tack value of the adhering water-soluble film. Various plasticisers can be used including, but by no means limited to, glycerol, sorbitol, polyethylene glycols and polypropylene glycols of various molecular weights, castor oil, and fish oil.

In the specific case of pharmaceutical active ingredients, the product may be applied to an to external surface lesion such as a burn or scald, an ulcer, to an internal wound, or alternatively to the mouth, vagina, rectum, or eye cavity, by which delivery system the active ingredient is slowly released into the area being treated while the product itself disintegrates or is bioabsorbed under the influence of its host environment, such that there is no need for its subsequent removal. Where nutraceutical ingredients are incorporated into the product, application via the mouth is preferred. Where sexual stimulants are incorporated into the product, application via the vagina is preferred.

Although the treatment of burns varies from hospital to hospital and from country to country, it is essential to prevent infection or if infection is already present, to bring this under control as rapidly as possible. This often involves the use of an anti-bacterial or an anti-fungal or an antibiotic composition. Among recommended active ingredients for the treatment of such infections are chlorhexidine, bacitracin, neomycin and polymixin, although these are by no means limiting. One of the many methods recommended for the treatment of burns is to apply a cream containing chlorhexidine and lignocaine which cream has the double advantage of being both antiseptic and pain relieving. Typically, for surface wounds, the dressing is left on for four or five days before being changed unless the wound becomes painful or there is any evidence of infection such as redness, swelling, or pain, when the dressing should be immediately changed. The use of this invention will facilitate observation of the area being treated since the product, unlike that of a typical dressing, will normally be almost transparent. However, where it is desirable to make the product opaque, any one or more of the water-soluble films comprising the product may be rendered opaque by loading with a filler such as a United States Pharmacopoeia (USP) grade of calcium carbonate.

In certain applications of the product described by this invention, it may be useful or indeed essential for air to reach the site being treated. In such cases, the product may be perforated throughout its entire thickness to a greater or lesser extent depending upon the desired amount of the surface requiring exposure to air.

The manufacture of the carrier water-soluble film with active ingredients embedded within or coated upon it follows the same principles as in the current practice of incorporating fillers. Those ingredients that are not water sensitive and any other additives such as plasticisers are mixed into the aqueous solution made from one or more selected water-soluble resins and then the resulting solution or suspension is cast upon a heated drum, upon a conveyor belt or upon a detachable liner, and then progressively dried to produce what is herein described as a cast film.

Ingredients that are water sensitive can be embedded into what is herein described as an extruded film, whether blown or die cast, by mixing the active ingredients into the pre-melted resin pellets prior to extrusion in the same way as are other additives such as plasticisers and slip agents.

In the case of heat sensitive ingredients, the cast film manufacturing process is preferred, whilst in the case of water-sensitive active ingredients, the extruded film manufacturing process is preferred.

Ingredients can be applied to the surface of the water-soluble carrier film either as an overall coating, or as a specific pattern by any of the current coating or printing methods used to print films. These ingredients may be applied to the carrier water-soluble film as solutions or dispersions in water or in any alternative liquid medium which will adhere to the water-soluble carrier film when dry and which is not detrimental to the film or to the active ingredients or to the site to be treated.

An indicator may be strategically placed on or within the product to indicate the precise location of the ingredient within the product. This may be done either by incorporating an indicator such as a colour marker (although this type of marker is by no means limiting) within the product in admixture with the ingredient or upon the product in a suitable ink which is then applied, for example by printing, in order to provide an indication visible to the naked eye of the location of the ingredient such that the product can subsequently be accurately positioned at the site in order that the ingredient is accurately targeted to the required area. In this way, wastage of what may be expensive ingredient(s) can be significantly reduced.

In a second aspect relating to the treatment of open wounds or other sites visible to the naked eye, an indicator such as a colour marker (although this type of marker is by no means limiting) may be incorporated within the product in admixture with the ingredient, and then used to measure the rate at which the ingredient is being dispensed. For example, in the case of a colour marker, the intensity of the colour will reduce as the ingredient is dispensed. By this means, the indicator, whether indicating by colour or by other means, can inform the patient or the operator that this stage of treatment is complete, allowing application of further product to be made at the appropriate time, if required.

Typically, the product is initially prepared as a web of material, and can subsequently either be presented as small, patch like, portions or slit into smaller convenient rolls of material. These presentations are, however, not limiting and other presentations may be considered. The web of material may be cut and processed either into rolls or into patches by any of the current methods used for producing paper, filmic or woven labels. The resulting patches can be of any shape or size and can be produced as individual pre-cut units or produced at the time of application by cutting to required size from a roll or sheet of product using, for example, a pair of scissors. The pre-cut form may be most suited to those surfaces that are similar in size, for example, surfaces within the mouth, vagina, rectum or eye cavity, and the sheet or roll form may be more suited for internal or external wound or burn sites that may vary considerably in size. Similarly, whilst the most convenient shape would be round or oval, it will be readily appreciated that whether pre-cut or cut from roll or sheet form, any shape of patch can be used, the only differences being those of convenience. The product is then applied to the site to be treated.

As an example of how the product may be used on an open wound, a roll is unwound and wrapped around an open wound or burn with the adhering water-soluble film facing the wound or burn. In this example, the product serves two purposes. It administers the active ingredient to the required area of open wound during a prescribed period of time by selecting a certain material for the carrier water-soluble film and a certain design of perforated protecting water-soluble film, and it excludes air from the wound helping to reduce the risk of infection. In comparison with the present method of application by cream or gel preparations, the dose rate provided in this example is uniform across the open wound and the weight of active ingredient can be adjusted to minimise any pain associated with its application. As the water-soluble film is absorbed over time into the wound, any surplus product is removed by cutting with a pair of scissors or other methods and fresh film is applied to the wound without the need to disturb previous applications, thereby allowing the wound to heal more quickly. Alternatively, whilst not disturbing the healing process, excess product can be removed by washing the wound with warm water. Different active ingredients can be applied during the healing period either by means of different rolls of product or by a combination of different ingredients incorporated within a single roll of product.

The following examples, although by no means limiting, will serve as illustrations to describe how the product may be used in surgical interventions.

In general surgery, a surgical wound which appears to be infected is operated on in order to clean the area. During surgery, it is customary to treat the area with locally applied antibiotics. Application during surgery of the product, which is prepared to deliver the necessary therapeutic dose and duration of treatment, will release antibiotics over an extended period of time and be beneficial to the treatment.

In abdominal surgery, as with appendix flabitis which is usually treated as infected, long term antibiotics are given intravenously. However, during surgery, the product described in this invention can be inserted in order to improve infiltration of the antibiotics due to the proximity of the product, even when laparoscopy is concerned, requiring not more than three incisions of 1.5 cm each.

In open fracture operations, the treatment is undoubtedly a procedure leading to potential infection. It will be evident that there are clear advantages resulting from use of the product to provide local antibiotic treatment over an extended period of time and which does not require replacement and avoids spreading of the infection from the affected area.

In a further embodiment of the invention, the product is used as a web of material to encapsulate other ingredients in addition to those contained within its mass. According to the selection of the most appropriate machinery to carry out the encapsulation process, one, two or even more webs of product according to this invention are required. Once the capsules have been filled, whether with powder, granules, gels or liquids, they are sealed by solvent welding, heat welding, radio frequency welding, laser welding or any other commonly used methods for sealing filmic materials. In this way, further alternative drug delivery systems are made possible by means of this invention.

In a second aspect, the invention provides a method of making the product according to the first aspect of the invention, the method comprising the steps of embedding or coating at least one ingredient in a water soluble carrier film, providing a water soluble protective film on each side of the carrier film, the protective films being less readily water soluble than the carrier film, perforating at least one of the protective films and providing a water soluble adhering film over the perforated protective film.

The steps may be carried out in any order to produce the product.

In a third aspect, the invention provides a method of using a product according to first aspect of the invention to apply one or more ingredients to a site in a controlled manner by applying the product to the site such that the carrier film gradually dissolves to release the ingredients through the perforated protective layer.

In a fourth aspect, the invention provides a dosing system for releasing one or more ingredients in a controlled manner to a site on a human or animal body, comprising embedding or coating at least one ingredient in or on a water soluble carrier film provided on each side with a water soluble protective film that is less readily water soluble than the carrier film, wherein one of the protective films is perforated for delivery of the at least one ingredient through the perforations to a site to be treated.

As will be apparent from the foregoing, the dosing system described herein provides a measured prescribed dose of one or more ingredients to a site at which an aqueous medium is present.

The invention will now be described in more detail by way of example only with reference to the accompanying drawings wherein:

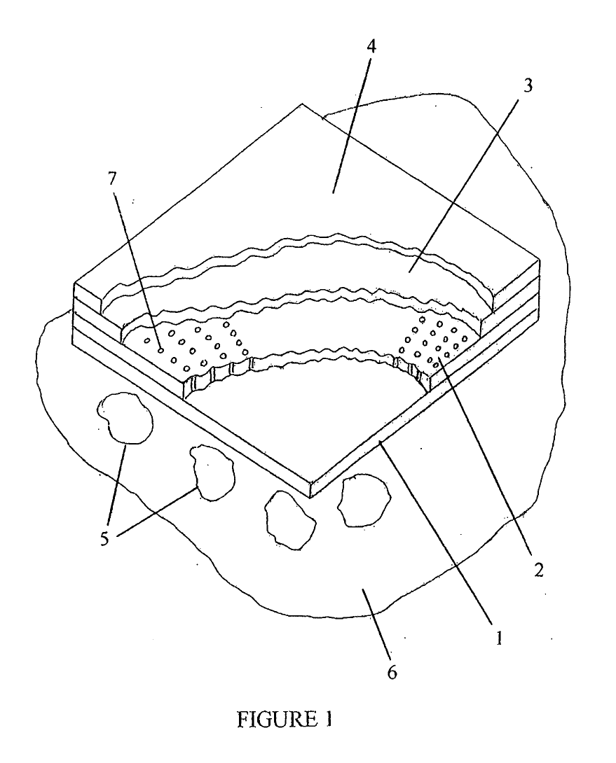

FIG. 1 is a perspective view of a first embodiment of the invention;

FIG. 2 is a perspective view of a second embodiment of the invention; and

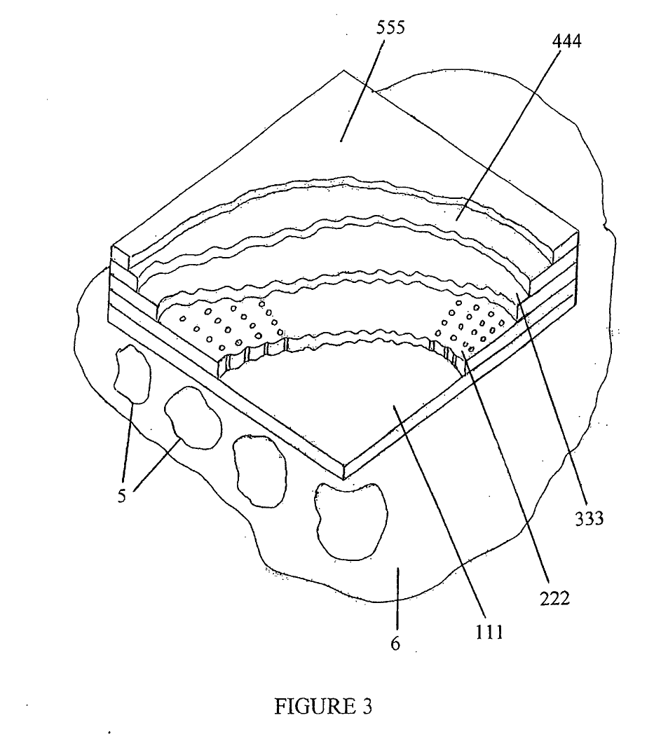

FIG. 3 is a perspective view of a third embodiment of the invention.

In FIG. 1, there is shown a first embodiment of the invention in which there is a single carrier water-soluble film (3) within which is embedded or upon which is coated one or more ingredients to be delivered. On either side of the carrier water-soluble film (3) are laminated protecting water-soluble films (2) and (4). One of the two protecting water-soluble films (2) is perforated in order to allow the ingredients to seep through the perforations (7). A further adhering water-soluble film (1) is laminated to the perforated protecting water-soluble film (2) in order to seal the perforations (7) and also to provide a layer which will adhere to a site (6) to be treated as soon as the product is brought into contact with an aqueous medium (5) on the surface of the site (6).

In FIG. 2, there is shown a second embodiment of the invention in which there are two carrier water-soluble films (31) and (32) both capable of delivering one or more ingredients. It will be evident that in this product construction, the size and/or number of perforations in protecting water-soluble film (21) can be larger than those in protecting water-soluble film (22) in order that the delivery of ingredients to the site is controlled by protecting perforated water-soluble film shown at (22). Alternatively, the protecting perforated water-soluble film (22) can be dispensed with such that carrier films 31 and 32 are laminated directly together. It will be noted that in this embodiment, the dissolution rate of carrier water-soluble film (31) can be more rapid than that of water-soluble carrier film (32).

In FIG. 3, there is shown a embodiment of the invention in which the unperforated protecting water-soluble film (444) has been further laminated to a detachable liner (555), preferably a printed detachable liner, in order that the location of the ingredients within the product is clearly visible to the user and hence the product can be accurately applied to the site so that delivery of the ingredient is optimised. The detachable liner (555) can be a paper liner or a polymeric liner.

The following examples describe processes for manufacturing the products illustrated in FIGS. 1 to 3.

### EXAMPLE 1

### One Water-Soluble Carrier Film (3)

Unperforated protecting water-soluble film (4)
Carrier water-soluble film (3) delivering one or more ingredients
Perforated protecting water-soluble film (2)
Adhering water-soluble film (1)
Aqueous Medium on surface
Site to be treated

1. We cast on a detachable liner a water soluble film dissolving at a temperature ranging from 35° C. to 65° C., preferably from 40° C. to 55° C., more preferably from 45° C. to 50° C.
2. We delaminated the detachable liner.
3. We perforated the water soluble film.
4. We cast on another detachable liner a water soluble film (to make sheet A) dissolving at a temperature ranging from 35° C. to 65° C., preferably from 40° C. to 55° C., more preferably from 45° C. to 50° C.
5. We cast an aqueous water soluble film forming composition loaded with neomycin powder on the water soluble film side of sheet A and simultaneously laminated the perforated water soluble film as received from step 3 to the neomycin loaded water soluble film while it was in semi-dried condition, to form sheet B. The moisture content in the semi dried water soluble film ranges from 20% to 60%, preferably from 25 to 50% more preferably from 28 to 40% and the film dissolves at a temperature ranging from 5° C. to 35° C., preferably from 10° C. to 30° C., more preferably from 15° C. to 20° C.
6. We detached the liner as used in step 4 to form sheet C.
7. We laminated a water soluble film dissolving at a temperature ranging from 5° C. to 35° C., preferably from 10° C. to 30° C., more preferably from 15° C. to 20° C., either plain or embossed, on top of the perforated water soluble film side of sheet C to form sheet D.
8. A cut portion of sheet D containing neomycin is then placed on the wound or the site to be treated, taking care to ensure that the adhering water-soluble film is facing the wound or affected area.

### EXAMPLE 2

### Two Water-Soluble Carrier Films (31 and 32)

Unperforated protecting water-soluble film (44)
Carrier water-soluble film (32) delivering one or more ingredients
Protecting perforated water-soluble film (22)
Carrier water-soluble film (31) delivering one or more ingredients
Protecting perforated water-soluble film (21)
Adhering water-soluble film (11)
Aqueous Medium on surface
Site to be treated

1. We cast on a detachable liner a water soluble film dissolving at a temperature ranging from 35° C. to 65° C., preferably from 40° C. to 55° C., more preferably from 45° C. to 50° C.
2. We delaminated the detachable liner.
3. We perforated the water soluble film.
4. We cast on another detachable liner a water soluble film (to make sheet A) dissolving at a temperature ranging from 35° C. to 65° C., preferably from 40° C. to 55° C., more preferably from 45° C. to 50° C.
5. We cast an aqueous water soluble film forming composition loaded with vancomycin powder on the water soluble film side of sheet A and simultaneously laminated perforated water soluble film as received from step 3 to the vancomycin loaded water soluble film while it was in semi-dried condition, to form sheet B. The moisture content in the semi dried water soluble film ranges from 20% to 60%, preferably from 25 to 50% more preferably from 28 to 40% and the film dissolves at a temperature ranging from 5° C. to 35° C., preferably from 10° C. to 30° C., more preferably from 15° C. to 20° C.
6. We detached the liner as used in step 4 to form sheet C.
7. We cast a water soluble film, dissolving at a temperature ranging from 5° C. to 35° C., preferably from 10° C. to 30° C., more preferably from 15° C. to 20° C., on another detachable liner and simultaneously laminated the perforated water soluble film as received from step 3 to the water soluble film while it was in semi-dried condition to form sheet D. The moisture content in the semi dried water soluble film ranges from 20% to 60%, preferably from 25 to 50% more preferably from 28 to 40%.
8. We cast an aqueous water soluble film forming composition loaded with another active ingredient or the same active ingredient on the perforated water soluble film side of sheet D and simultaneously laminated the perforated water soluble film side of sheet C to the active ingredient loaded water soluble film while it was in semi-dried condition, to form sheet E. The moisture content in the semi dried water soluble film ranges from 20% to 60%, preferably from 25 to 50% more preferably from 28 to 40%.
9. A cut portion of sheet E containing one or more active ingredients is then placed on the wound or the site to be treated, taking care to ensure that the adhering water-soluble film is facing the wound or affected area.

In the same way, by repeating the above process, any number of water soluble layers can be laminated to carry several or same types of active ingredients.

### EXAMPLE 3

### One Water-Soluble Carrier Film (333) with Release Liner

Detachable liner (555)
Unperforated protecting water-soluble film (444)
Carrier water-soluble film (333) delivering one or more ingredients
Protecting perforated water-soluble film (222)
Adhering water-soluble film (111)
Aqueous Medium on surface
Site to be treated

1. We cast on a detachable liner a water soluble film dissolving at a temperature ranging from 35° C. to 65° C., preferably from 40° C. to 55° C., more preferably from 45° C. to 50° C.
2. We delaminated the detachable liner.
3. We perforated the water soluble film.
4. We cast on another detachable liner a water soluble film (to make sheet A) dissolving at a temperature ranging from 35° C. to 650C, preferably from 40° C. to 55° C., more preferably from 45° C. to 50° C. The detachable liner is printed, in order that the location of the ingredients within the product is clearly visible to the user and hence the product can be accurately applied to the site so that delivery of the ingredient is optimised.
5. We cast an aqueous water soluble film forming composition loaded with neomycin powder on the water soluble film side of sheet A and simultaneously laminated the perforated water soluble film as received from step 3 to the neomycin loaded water soluble film while it was in semi-dried condition, to form sheet B. The moisture content in the semi dried water soluble film ranges from 20% to 60%, preferably from 25 to 50% more preferably from 28 to 40% and the film dissolves at a temperature ranging from 5° C. to 35° C., preferably from 10° C. to 30° C., more preferably from 15° C. to 20° C.
6. We laminated a water soluble film, dissolving at a temperature ranging from 5° C. to 35° C., preferably from 10° C. to 30° C., more preferably from 15° C. to 20° C., either plain or embossed, on top of the perforated water soluble film side of sheet B to form sheet C.
7. A cut portion of sheet C containing one or more active ingredients is then placed on the wound or the site to be treated, taking care to ensure that the adhering water-soluble film is facing the wound or affected area.

In the above-described examples, the water soluble materials forming the protective films are chosen to be less readily water soluble at a given temperature than the water soluble materials forming the carrier films and the water soluble materials forming the adhering layer while the solubility of the water soluble materials forming the carrier films at a given temperature may be chosen to be similar to that of the water soluble materials forming the adhering film. Thus, in use of the products, the adhering film and carrier film(s) will tend to dissolve more readily at a given temperature than the protecting film(s) although the latter may eventually dissolve over an extended period of time so that there is no need for subsequent physical removal of the products from the site.

1. A water-soluble or water-dispersible product containing one or more ingredients in which the ingredient is embedded within or coated upon one or more carrier water soluble films, which are protected by less readily soluble water soluble films on either side at least one of which protective films is perforated to allow the delivery of ingredient through the perforations when the product is placed in contact with an aqueous medium at an appropriate site, thereby allowing the ingredient to be dispensed and the product at least partially dissolved or dispersed over a period of time such that there is no need for its subsequent physical removal from the site.

2. A product according to claim 1 in which the product includes an adhering water soluble film that self adheres to at least a part of the site due to solubilisation of part or all of the adhering water-soluble film following contact with an aqueous medium present at the site.

3. (canceled)

4. A product according to claim 1 in which the product is bioabsorbable.

5. A product according to claim 1 in which at least one of the one or more water-soluble or water-dispersible films are made from starch-based materials.

6. A product according to claim 1 in which the one or more ingredients are pharmaceutical or nutraceutical active ingredients for use in man or in animals.

7. A product according to claim 6 in which the ingredients are an anti-bacterial or an anti-fungal or an antibiotic composition or a mixture thereof.

8. A product, according to claim 1 in which the rate of release of the required ingredients is controlled by the rate of solubilisation of the carrier water-soluble film.

9. A product, according to claim 1 in which the rate of release of the required ingredients is controlled by the size and number of perforations in the perforated protecting water-soluble film or films.

10. A product according to claim 1 in which different ingredients are located within the product in such a way that they are released sequentially to the site.

11. A product according to claim 1 comprising a plurality of water soluble carrier films.

12. A product according to claim 11 wherein adjacent water soluble carrier films are separated by a perforated water soluble protecting film.

13. A product according to claim 11 wherein adjacent water soluble carrier films directly contact each other.

14. A product according to claim 11 wherein different ingredients are contained in different water soluble carrier films.

15. A product according to claim 1 in which a dye or other indicator is incorporated ingredients in order to identify that part of the product containing the ingredients.

16. (canceled)

17. A product according to claim 1 in which a dye or other indicator is incorporated in admixture with the one or more ingredients in order to monitor the rate at which the one or more ingredients are being dispensed.

18. A product according to claim 1 in which one or more ingredients are applied in a pattern to the product in order to obtain greater precision of dosage release and reduced wastage of the ingredients.

19. (canceled)

20. (canceled)

21. (canceled)

22. A product according to claim 1 in which the protecting water soluble film on the side of the carrier film opposite the perforated protecting layer is laminated to a printed detachable liner.

23. A product according to claim 22 in which the printed detachable liner identifies that part of the product within which the ingredient or ingredients have been embedded or upon which the ingredient or ingredients have been coated such that the person applying the product shall be able to position the product accurately at the site and then detach the printed liner.

24. A method of making a product according to My preceding claim 1 comprising carrying out in any order the steps of embedding or coating the at least one ingredient in a water soluble carrier film, providing a water soluble protective film on each side of the carrier film, the protective films being less readily water soluble than the carrier film, perforating at least one of the protective films and providing a water soluble adhering film over the perforated protective film.

25. (canceled)

26. (canceled)

27. A dosing system for releasing one or more ingredients in a controlled manner to a site on a human or animal body, comprising embedding or coating at least one ingredient in a water soluble carrier film provided on each side with a water soluble protective film that is less readily water soluble than the carrier film, wherein one of the protective films is perforated for delivery of the at least one ingredient through the perforations to a site to be treated.
